(19) United States
(12) Patent Application Publication
(10) Pub. No.: US 2024/0076796 A1
(43) Pub. Date: Mar. 7, 2024

(54) PULSE METHOD FOR THE ELECTRODEPOSITION OF CONDUCTIVE POLYMER

(71) Applicant: Heraeus Medevio GmbH & Co. KG, Hanau (DE)

(72) Inventor: Leonard STOICA, Hanau (DE)

(21) Appl. No.: 18/460,036

(22) Filed: Sep. 1, 2023

(30) Foreign Application Priority Data
Sep. 5, 2022 (DE) 102022122416.3

(51) Int. Cl. C25D 21/12; A61B 5/268; A61N 1/05; C25D 9/02

(52) U.S. Cl. CPC C25D 21/12; A61B 5/268; A61N 1/05; C25D 9/02; A61B 2562/125

(57) ABSTRACT

The present invention relates to a method for producing a layered body, wherein the layered body comprises an electrically conductive substrate, the surface of which is at least partially coated with a layer containing an electrically conductive polymer. The invention also relates to a layered body obtainable by this method, to a layered body comprising an electrically conductive substrate and a layer containing an electrically conductive polymer, which layer is deposited on at least a part of the surface of the electrically conductive substrate, to a medical device and to the use of a layered body.

### CROSS-REFERENCES TO RELATED REFERENCES

This application claims priority pursuant to 35 U.S.C. 119(a) to German Application No. 102022122416.3, filed Sep. 5, 2022, which application is incorporated herein by reference in its entirety.

### DESCRIPTION

The present invention relates to a method for producing a layered body by electrodeposition, to a layered body obtainable by this method, to a layered body comprising an electrically conductive substrate and a layer containing an electrically conductive polymer, which layer is deposited on at least a part of the surface of the electrically conductive substrate, to a medical device and to the use of a layered body therein.

Electrically conductive polymers are used nowadays in electronics, in medical technology and in general industry. In medical technology, they are applied, for example, as an electrically conductive coating to electrodes, which can in particular also be provided to be implanted in the human body in order to detect electrophysiological signals in a tissue or to electrically stimulate a tissue.

However, it has been found that electrically conductive polymers, when applied as coatings on substrates, show an adhesion to the substrate that is insufficient for some applications. For example, it has been found that coatings of electrochemically deposited poly(3,4-ethylenedioxythiophene) (PEDOT) with polystyrene sulfonate (PSS) as the counterion can be mechanically stiff and brittle, and tend to crack and detach under load.

In order to address this problem, WO 2015/031265 A1 proposes adding photoreactive anionic polymer compounds and further photoreactive compounds, such as photoreactive crosslinkers, to the monomer composition used during electrodeposition, and exposing the PEDOT-containing layer obtained after electrodeposition to UV radiation in order to cause a reaction of the photoreactive components. A monomer composition suitable in this context is available from Heraeus Medical Components, Germany, under the name “Amplicoat®”.

It has been found, however, that when using a monomer composition such as, for example, “Amplicoat®” in conventional electrodeposition methods, electrically conductive coatings are obtained which are still in need of improvement in particular with regard to the homogeneity of the layer thickness and the surface morphology.

The object of the present invention was therefore that of overcoming, or at least reducing the scale of, the disadvantages of the prior art electrodeposition of electrically conductive polymers on electrically conductive substrates, in particular in connection with the use of monomer compositions such as, for example, “Amplicoat®” in such electrodeposition methods.

The object of the present invention was, in particular, that of providing an electrodeposition method that can be used to deposit electrically conductive polymers on substrate surfaces to obtain electrically conductive layers that are characterized by particularly high layer thickness homogeneity and by particularly low roughness on the surface of the electrically conductive layer. Furthermore, these deposited electrically conductive layers should be characterized by particularly good adhesion to the substrate surface and, at the same time, by electrical properties which are as good as possible, such as impedance and charge storage capacity.

The subject matter of the category-forming claims contributes to achieving at least one of these objects, with subsidiary and dependent claims constituting preferred embodiments of the present invention.

|1a| A contribution to achieving at least one of the above-mentioned objects is made by a 1st embodiment of a method for producing a layered body, preferably a layered body in the form of an electrode or part of an electrode, particularly preferably in the form of an electrode or part of an electrode for detecting electrophysiological signals in a tissue or in part of a tissue or for electrically stimulating a tissue or part of a tissue, even more preferably in the form of an implantable electrode or part of an implantable electrode, wherein the layered body comprises an electrically conductive substrate, the surface of which is at least partially coated with a layer containing an electrically conductive polymer, the method comprising the following method steps:
a) providing the electrically conductive substrate;
b) contacting at least a part of the surface of the electrically conductive substrate with a composition containing
   at least one monomer suitable for producing an electrically conductive polymer,
   at least one solvent,
   optionally at least one crosslinker, preferably a photoreactive crosslinker;
c) applying an electrical potential between the electrically conductive substrate and the composition such that the at least one monomer i) polymerizes to form an electrically conductive polymer, and the electrically conductive polymer formed is deposited in the form of a layer containing this polymer on at least a part of the surface of the electrically conductive substrate;
wherein applying the electrical potential comprises a pulse phase PP having successive pulses, wherein each pulse of the pulse phase PP comprises a partial pulse phase PP₁ having a phase duration t₁ and a partial pulse phase PP₂ having a phase duration t₂,
wherein the partial pulse phase PP₁ is characterized by an electrical potential EP(PP₁) and by a current density SD(PP₁) guided through the electrically conductive substrate;
wherein the partial pulse phase PP₂ is characterized by an electrical potential EP(PP₂) and by a current density SD(PP₂) guided through the electrically conductive substrate;
   wherein EP(PP₁)<EP(PP₂) and/or SD(PP₁)<SD(PP₂);
d) optionally contacting the layer containing an electrically conductive polymer, which layer was obtained in method step c), with at least one crosslinker, preferably a photoreactive crosslinker;
e) optionally applying electromagnetic radiation to the layer containing the electrically conductive polymer in order to cause a photoreaction of the at least one of the photoreactive crosslinkers iii).

Preferably, the coating with the electrically conductive polymer is arranged in the layered body obtained by the method according to the invention (and also in the below-described layered body 1 and 2 according to the invention) such that, when used during a use of this layered body as an electrode or as part of an electrode for detecting electrophysiological signals in a tissue or in a part of a tissue or for electrically stimulating a tissue or a part of a tissue, the coating comes into contact, preferably into direct contact, with the tissue or the part of the tissue.

If the composition used in method step b) already contains a photoreactive crosslinker iii), it is preferred according to the invention that although no method step d) is performed, a method step e) takes place. Conversely, it is also preferred for method step d) to be performed only if the composition used in method step b) does not contain a photoreactive crosslinker.

Furthermore, the method comprises configurations in which each pulse begins with partial pulse phase PP₁, followed by partial pulse phase PP₂, and configurations in which each pulse begins with partial pulse phase PP₂, followed by partial pulse phase PP₁.

Furthermore, the method also comprises configurations in which the pulse phase PP comprises pulses for which EP(PP₁)<EP(PP₂) as well as pulses for which SD(PP₁)<SD(PP₂). However, configurations of the method in which either EP(PP₁)<EP(PP₂) for all pulses of the pulse phase PP or SD(PP₁)<SD(PP₂) for all pulses are particularly preferred according to the invention.

|2a| According to a preferred embodiment of the method for producing a layered body, EP(PP₁)<EP(PP₂), wherein, during partial pulse phase PP₁ and partial pulse phase PP₂, the electrical potential is substantially constant, or is particularly preferably constant, within the relevant partial pulse phase. A “substantially constant electrical potential” within partial pulse phase PP₁ or within partial pulse phase PP₂ exists when the electrical potential, at any time within partial pulse phase PP₁ or within partial pulse phase PP₂, deviates by no more than 1%, preferably by no more than 0.5% and most preferably by no more than 0.1%, from an electrical potential EP_{Reference}, as determined by a reference electrode, in partial pulse phase PP₁ or in partial pulse phase PP₂. Such a method is also referred to as “pulse potentiostatic method.”

This preferred embodiment is a 2nd embodiment of the method according to the invention, which is preferably dependent on the 1st embodiment.

|3a| According to a further preferred embodiment of the method for producing a layered body, the electrical potential EP(PP₁) during partial pulse phase PP₁ is in a range of 0.2 to 0.8 V, preferably in a range of 0.3 to 0.7 V, more preferably in a range of 0.4 to 0.6 V, and most preferably in a range of 0.45 to 0.55 V, and the electrical potential EP(PP₂) during partial pulse phase PP₂ is in a range of 0.8 to 1.4 V, preferably in a range of 0.9 to 1.3 V, more preferably in a range of 1.0 to 1.2 V, and most preferably in a range of 1.05 to 1.15 V. This preferred embodiment is a 3rd embodiment of the method according to the invention, which is preferably dependent on the 2nd embodiment.

|4a| According to yet another preferred embodiment of the method for producing a layered body, t₁ is in a range of 1 to 15 sec, preferably in a range of 4 to 13 sec, more preferably in a range of 8 to 12 sec, and most preferably in a range of 9 to 11 sec, and t₂ is in a range of 1 to 10 sec, preferably in a range of 2 to 8 sec, more preferably in a range of 3 to 7 sec, and most preferably in a range of 4 to 6 sec. This preferred embodiment is a 4th embodiment of the method according to the invention, which is preferably dependent on the 2nd or 3rd embodiment.

|5a| According to a further preferred embodiment of the method for producing a layered body, SD(PP₁)<SD(PP₂), wherein, during partial pulse phase PP₁ and partial pulse phase PP₂, the current density guided through the electrically conductive substrate is substantially constant, particularly preferably constant, within the relevant partial pulse phase. A “substantially constant current density guided through the substrate” within partial pulse phase PP₁ or within partial pulse phase PP₂ exists when the current density guided through the substrate, at any time within partial pulse phase PP₁ or within partial pulse phase PP₂, deviates by no more than 5%, preferably by no more than 1% and most preferably by no more than 0.1%, from the mean value of the current density guided through the substrate in partial pulse phase PP₁ or in partial pulse phase PP₂. Such a method is also referred to as “pulse galvanostatic method.” This preferred embodiment is a 5th embodiment of the method according to the invention, which is preferably dependent on the 1st embodiment.

|6a| According to a further preferred embodiment of the method for producing a layered body, the amount of the current density |SD(PP₁)| guided through the electrically conductive substrate during partial pulse phase PP₁ is less than 0.4 mA/cm², preferably less than 0.3 mA/cm², more preferably less than 0.2 mA/cm² and most preferably less than 0.1 mA/cm², (wherein |SD(PP₁)| can, in particular, also assume a zero value in partial pulse phase PP₁), and the amount of the current density 1SD(PP₂)| guided through the electrically conductive substrate during partial pulse phase PP₂ is in a range of 0.9 to 1.7 mA/cm², preferably in a range of 1 to 1.6 mA/cm², more preferably in a range of 1.1 to 1.5 mA/cm² and most preferably in a range of 1.2 to 1.4 mA/cm². It is furthermore preferred in this context that, if the current density guided through the electrically conductive substrate in partial pulse phase PP₁ and partial pulse phase PP₂ is not equal to zero, the current density guided through the electrically conductive substrate has a positive value (i.e., that an oxidation process takes place in which the monomer i) suitable for producing an electrically conductive polymer is converted into a radical). This preferred embodiment is a 6th embodiment of the method according to the invention, which is preferably dependent on the 5th embodiment.

|7a| According to yet another preferred embodiment of the method for producing a layered body, t₁ is in a range of 1 to 15 sec, preferably in a range of 5 to 13 sec, more preferably in a range of 8 to 12 sec, and most preferably in a range of 9 to 11 sec, and t₂ is in a range of 1 to 10 sec, preferably in a range of 2 to 8 sec, more preferably in a range of 3 to 7 sec, and most preferably in a range of 4 to 6 sec. This preferred embodiment is a 7th embodiment of the method according to the invention, which is preferably dependent on the 5th or 6th embodiment.

|8a| According to a further preferred embodiment of the method for producing a layered body, the method comprises a method step cₒ) in which, before the beginning of pulse phase PP, for duration t₀, the electrical potential EP₀ or the current density SD₀ guided through the electrically conductive substrate is substantially constant, preferably constant (substrate-electrolyte equilibration phase). In the case of the “pulse potentiostatic method,” the electrical potential EP₀ in this method step cₒ) can also be constant. However, it is also possible to set a constant current density SDₒ guided through the electrically conductive substrate in this method step cₒ). Conversely, in the case of the “pulse galvanostatic method,” the current density SD₀ guided through the electrically conductive substrate can also be constant. Analogously, it is however also possible here to set a constant electrical potential EP₀ in this method step cₒ). This preferred embodiment is an 8th embodiment of the method according to the invention, which is preferably dependent on an embodiment selected from the 1st to 7th embodiment.

|9a| According to a further preferred embodiment of the method for producing a layered body, the amount of the current density |SD₀ guided through the electrically conductive substrate is less than 0.4 mA/cm², preferably less than 0.3 mA/cm², more preferably less than 0.2 mA/cm² and most preferably less than 0.1 mA/cm² (wherein |SD₀| can assume a zero value here as well). This preferred embodiment is a 9th embodiment of the method according to the invention, which is preferably dependent on the 8th embodiment.

|10a| According to a further preferred embodiment of the method for producing a layered body, the electrical potential EP₀ is in a range of 0.2 to 0.8 V, preferably in a range of 0.3 to 0.7 V, more preferably in a range of 0.4 to 0.6 V, and most preferably in a range of 0.45 to 0.55 V. This preferred embodiment is a 10th embodiment of the method according to the invention, which is preferably dependent on the 8th embodiment.

|11a| According to a further preferred embodiment of the method for producing a layered body, t₀ is in a range of 1 to 15 sec, preferably in a range of 4 to 13 sec, more preferably in a range of 8 to 12 sec, and most preferably in a range of 9 to 11 sec. This preferred embodiment is an 11th embodiment of the method according to the invention, which is preferably dependent on an embodiment selected from the 8th to 10th embodiment.

|12a| According to a further preferred embodiment of the method for producing a layered body, the pulse phase PP comprises at least 2, preferably at least 4, even more preferably at least 6, even more preferably at least 8 and most preferably at least 10 pulses. In a first particular configuration of this embodiment, the pulse phase PP can comprise 8 to 16 pulses, preferably 10 to 14 pulses and particularly preferably 13 to 15 pulses. According to a second particular configuration of this embodiment, it can comprise 20 to 28 pulses, preferably 22 to 26 pulses and particularly preferably 23 to 25 pulses. According to a third particular configuration of this embodiment, it can comprise 32 to 40 pulses, preferably 34 to 38 pulses and particularly preferably 35 to 37 pulses. This preferred embodiment is a 12th embodiment of the method according to the invention, which is preferably dependent on an embodiment selected from the 1st to 11th embodiment.

|13a| According to a further preferred embodiment of the method for producing a layered body, the individual pulses of pulse phase PP follow one another such that partial pulse phase PP₁ is followed by a partial pulse phase PP₂ and partial pulse phase PP₂ is followed by a partial pulse phase PP₁, preferably directly in each case. The following then applies to the total duration of a single pulse tₚ during pulse phase PP: tₚ=t₁=t₂. This preferred embodiment is a 13th embodiment of the method according to the invention, which is preferably dependent on an embodiment selected from the 1st to 12th embodiment.

|14a| According to a further preferred embodiment of the method for producing a layered body, the pulse phase PP has a total duration of 30 sec to 20 min, preferably of 1 min to 10 min, and most preferably of 2 min to 6 min. This preferred embodiment is a 14th embodiment of the method according to the invention, which is preferably dependent on an embodiment selected from the 1st to 13th embodiment.

|15a| According to a further preferred embodiment of the method for producing a layered body, the electrically conductive substrate is selected from the group consisting of gold, nickel, cobalt, iridium, titanium, niobium, tungsten, steel, platinum, silicon and alloys of at least two of these metals. This preferred embodiment is a 15th embodiment of the method according to the invention, which is preferably dependent on an embodiment selected from the 1st to 14th embodiment.

|16a| According to a further preferred embodiment of the method for producing a layered body, the electrically conductive substrate is selected from the group consisting of gold, platinum and a PtIr alloy. This preferred embodiment is a 16th embodiment of the method according to the invention, which is preferably dependent on the 15th embodiment.

|17a| According to a further preferred embodiment of the method for producing a layered body, the monomer i) suitable for producing an electrically conductive polymer is selected from the group consisting of a thiophene or a derivative thereof, pyrrole or a derivative thereof, aniline or a derivative thereof and a combination of at least two of these monomers. This preferred embodiment is a 17th embodiment of the method according to the invention, which is preferably dependent on an embodiment selected from the 1st to 16th embodiment.

|18a| According to a further preferred embodiment of the method for producing a layered body, the monomer i) suitable for producing an electrically conductive polymer is selected from the group consisting of 3,4-ethylenedioxythiophene (EDOT), hydroxymethyl-EDOT, EDOT-vinyl, EDOT-allyl ether, EDOT-COOH, EDOT-MeOH, EDOT-silane, EDOT-acrylate, EDOT-sulfonate, EDOT-amine, EDOT-amide, sodium 4-[(2,3-dihydrothieno[3,4-b][1,4]dioxin-2-yl)methoxy]butane-1-sulfonate (EDOT-S), sodium 4-[(2,3-dihydrothieno[3,4-b][1,4]dioxin-2-yl)methoxy]butane-2-sulfonate (EDOT-S), dimers or trimers of these thiophene monomers and combinations of at least two of these EDOT monomers. This preferred embodiment is an 18th embodiment of the method according to the invention, which is preferably dependent on an embodiment selected from the 1st to 17th embodiment.

|19a| According to a further preferred embodiment of the method for producing a layered body, the monomer i) suitable for producing an electrically conductive polymer is 3,4-ethylenedioxythiophene or a derivative thereof, particularly preferably 3,4-ethylenedioxythiophene. This preferred embodiment is a 19th embodiment of the method according to the invention, which is preferably dependent on the 18th embodiment.

|20a| According to a further preferred embodiment of the method for producing a layered body, the composition also contains, in addition to the EDOT monomers mentioned in the 18th and 19th embodiment, the corresponding ProDOT monomers (ProDOT=3,4-propylenedioxythiophene), preferably ProDOT monomers selected from the group consisting of ProDOT, hydroxymethyl-ProDOT, ProDOT-vinyl, ProDOT-allyl ether, ProDOT-COOH, ProDOT-MeOH, ProDOT-silane, ProDOT-silanol, ProDOT-acrylate, ProDOT-sulfonate, ProDOT-amine, ProDOT-amide, ProDOT-S, dimers or trimers of these ProDOT monomers and combinations of at least two of these ProDOT monomers. This preferred embodiment is a 20th embodiment of the method according to the invention, which is preferably dependent on the 18th or 19th embodiment.

|21a| According to a further preferred embodiment of the method for producing a layered body, the monomer i) suitable for producing an electrically conductive polymer is a mixture of 3,4-ethylenedioxythiophene (EDOT) and 3,4-propylenedioxythiophene (ProDOT), wherein the weight ratio of EDOT:ProDOT is preferably at least 4:1, particularly preferably at least 7:1, even more preferably at least 9:1 and most preferably at least 20:1. This preferred embodiment is a 21st embodiment of the method according to the invention, which is preferably dependent on an embodiment selected from the 1st to 20th embodiment.

|22a| According to a further preferred embodiment of the method for producing a layered body, the composition used in method step b) contains the monomer i) suitable for producing an electrically conductive polymer in a concentration in a range of 0.001 M to 1 M, preferably in a range of 0.01 M to 0.2 M. In the case of a mixture of two or more monomers i), the ranges mentioned above relate to the total concentration of monomers i). This preferred embodiment is a 22nd embodiment of the method according to the invention, which is preferably dependent on an embodiment selected from the 1st to 21st embodiment.

|23a| According to a further preferred embodiment of the method for producing a layered body, the composition used in method step b) comprises a photoreactive crosslinker iii). In this case, method step d) can be omitted. However, it is also possible to add a photoreactive crosslinker iii) to the composition used in method step b) and, additionally, to contact the layer obtained in method step c) containing an electrically conductive polymer with at least one photoreactive crosslinker iii) in method step d). This preferred embodiment is a 23rd embodiment of the method according to the invention, which is preferably dependent on an embodiment selected from the 1st to 22nd embodiment.

|24a| According to a further preferred embodiment of the method for producing a layered body, a photoreactive crosslinker iii), preferably the photoreactive crosslinker iii) which is included in the composition used in method step b), comprises at least one anionic photoreactive hydrophilic polymer containing polyacrylamide and photoreactive groups (=photoreactive polyacrylamide; “photo-PA”). This preferred embodiment is a 24th embodiment of the method according to the invention, which is preferably dependent on an embodiment selected from the 1st to 23rd embodiment.

|25a| According to a further preferred embodiment of the method for producing a layered body, the photoreactive crosslinker iii), preferably the photoreactive crosslinker iii) which is included in the composition used in method step b), comprises at least one anionic photoreactive hydrophilic polymer having sulfonate groups, preferably acrylamido-2-methylpropane sulfonate groups (AMPS groups). This preferred embodiment is a 25th embodiment of the method according to the invention, which is preferably dependent on an embodiment selected from the 1st to 24th embodiment.

|26a| According to a further preferred embodiment of the method for producing a

layered body, the photoreactive crosslinker iii), preferably the photoreactive crosslinker iii) which is included in the composition used in method step b), comprises at least one photoreactive polyacrylamide selected from the group consisting of N-acetylated poly[acrylamide-co-sodium-2-acrylamido-2-methylpropane sulfonate-co-N-(3-(4-benzoylbenzamido) propyl)methacrylamide]-co-methoxypoly(ethylene glycol) and poly[acrylamide-co-sodium-2-acrylamido-2-methylpropanesulfonate-co-N-(3-(4-benzoylbenzamido)propyl) methacrylamide]. Further suitable photoreactive polyacrylamides are described in U.S. Pat. Nos. 4,979,959, 5,263,992 and 5,512,329. This preferred embodiment is a 26th embodiment of the method according to the invention, which is preferably dependent on the 24th or 25th embodiment.

|27a| According to a further preferred embodiment of the method for producing a layered body, the composition used in method step b) comprises at least one anionic photoreactive hydrophilic polymer containing polyacrylamide and photoreactive groups as a photoreactive crosslinker iii) in a concentration in a range of 2.5 to 50 mg/ml, preferably in a range of 5 to 25 mg/ml. This preferred embodiment is a 27th embodiment of the method according to the invention, which is preferably dependent on an embodiment selected from the 24th to 26th embodiment.

|28a| According to a further preferred embodiment of the method for producing a layered body, the photoreactive crosslinker iii), preferably the photoreactive crosslinker iii) which is included in the composition used in method step b), comprises an anionic photoreactive crosslinking agent having sulfonate groups, carboxylate groups, phosphonate groups, phosphate groups or combinations of these groups. This preferred embodiment is a 28th embodiment of the method according to the invention, which is preferably dependent on an embodiment selected from the 1st to 27th embodiment.

|29a| According to a further preferred embodiment of the method for producing a layered body, the photoreactive crosslinker iii), preferably the photoreactive crosslinker iii) which is included in the composition used in method step b), comprises, as an anionic photoreactive crosslinking agent, a compound having the structure X₁-Y-X₂, in which X₁ and X₂ independently of one another represent a radical comprising a latent photoreactive group. These photoreactive groups can be, for example, an aryl ketone or a quinone. The radical Y ensures the desired water solubility of the anionic photoactivatable crosslinking agent. The water solubility (at room temperature and an optimal pH) of the anionic photoreactive crosslinking agent is preferably at least about 0.05 mg/ml. In the X₁-Y-X₂ structure, Y can be a radical having at least one acidic group or a salt thereof. Such a photoactivatable crosslinking agent can be anionic, depending on the pH of the composition. Suitable acidic groups are, for example, sulfonic acids, carboxylic acids, phosphonic acids and the like. Suitable salts of such groups are, for example, sulfonate salts, carboxylate salts and phosphate salts. The crosslinking agent can contain, for example, a sulfonic acid or sulfonate group. Suitable counterions are alkali metals and alkaline earth metals, ammonium, protonated amines and the like. Suitable anionic photoreactive crosslinking agents are described, for example, in U.S. Pat. No. 6,278,018 or US 2012/0046384 A1. This preferred embodiment is a 30th embodiment of the method according to the invention, which is preferably dependent on the 29th embodiment.

|31a| According to a further preferred embodiment of the method for producing a layered body, the photoreactive crosslinker iii), preferably the photoreactive crosslinker iii) which is included in the composition used in method step b), comprises, as an anionic photoreactive crosslinking agent, a compound selected from the group consisting of disodium 4,5-bis[(4-benzoylbenzyl)oxy]-1,3-benzenedisulfonate and sodium bis(4-benzoylphenyl)phosphate. This preferred embodiment is a 31st embodiment of the method according to the invention, which is preferably dependent on the 29th or 30th embodiment.

|32a| According to a further preferred embodiment of the method for producing a layered body, the photoreactive crosslinker iii), preferably the photoreactive crosslinker iii) which is included in the composition used in method step b), contains a photoreactive and uncharged hydrophilic polymer. This preferred embodiment is a 32nd embodiment of the method according to the invention, which is preferably dependent on an embodiment selected from the 1st to 31st embodiment.

|33a| According to a further preferred embodiment of a method for producing a layered body, the photoreactive crosslinker iii), preferably the photoreactive crosslinker iii) which is included in the composition used in method step b), contains, as a photoreactive and uncharged hydrophilic polymer, a photoreactive 1-vinyl-2-pyrrolidone derivative. This preferred embodiment is a 33rd embodiment of the method according to the invention, which is preferably dependent on the 32nd embodiment.

|34a| According to a further preferred embodiment of the method for producing a layered body, the photoreactive crosslinker iii), preferably the photoreactive crosslinker iii) which is included in the composition used in method step b), contains at least one anionic photoreactive hydrophilic polymer and at least one photoreactive and uncharged hydrophilic polymer in a weight ratio in a range of 1:10 to 10:1, preferably in a range of 1:2 to 2:1, even more preferably in a range of 1:1.5 to 1.5:1. This preferred embodiment is a 34th embodiment of the method according to the invention, which is preferably dependent on an embodiment selected from the 1st to 33rd embodiment.

|35a| According to a further preferred embodiment of the method for producing a layered body, the at least one solvent ii) is an aprotic organic solvent, a polar organic solvent or a mixture thereof. This preferred embodiment is a 35th embodiment of the method according to the invention, which is preferably dependent on an embodiment selected from the 1st to 34th embodiment.

|36a| According to a further preferred embodiment of the method for producing a layered body, the at least one solvent ii) is selected from the group consisting of acetonitrile, dichloromethane, dimethyl sulfoxides, acetone, dimethylformamide, isopropanol, methanol, ethanol, water and mixtures of at least two of these solvents. This preferred embodiment is a 36th embodiment of the method according to the invention, which is preferably dependent on the 35th embodiment.

|37a| According to a further preferred embodiment of the method for producing a layered body, the composition used in method step b) contains a surface-active compound iv). This preferred embodiment is a 37th embodiment of the method according to the invention, which is preferably dependent on an embodiment selected from the 1st to 36th embodiment.

|38a| According to a further preferred embodiment of the method for producing a layered body, the composition used in method step b) contains, as a surface-active compound iv), a compound selected from the group consisting of poloxamers, polyoxyethylene alkyl ethers, polysorbitan, polyoxyethylene derivatives of sorbitan monolaurate and mixtures of at least two of these compounds. This preferred embodiment is a 38th embodiment of the method according to the invention, which is preferably dependent on the 37th embodiment.

|39a| According to a further preferred embodiment of the method for producing a layered body, application of an electrical potential between the electrically conductive substrate and the composition in method step c) takes place in a device comprising the electrically conductive substrate as the working electrode, a counter electrode which preferably has an electrode surface that is at least 2 times, even more preferably at least 5 times and most preferably at least 10 times as large as the electrode surface of the working electrode, and optionally a reference electrode. This preferred embodiment is a 39th embodiment of the method according to the invention, which is preferably dependent on an embodiment selected from the 1st to 38th embodiment.

|40a| According to a further preferred embodiment of the method for producing a layered body, the electromagnetic radiation applied in method step e) is UV light in a wavelength range of 260 to 400 nm. This preferred embodiment is a 40th embodiment of the method according to the invention, which is preferably dependent on an embodiment selected from the 1st to 39th embodiment.

41a| According to a further preferred embodiment of the method for producing a layered body, the electrically conductive substrate provided in method step a) is purified prior to method steps b) and c), i.e. prior to the electrodeposition of the electrically conductive polymer, preferably by a purification method selected from the group consisting of purification with ultrasound, purification by chemical (acidic) etching, purification by means of alkaline cleaning agents, purification by plasma treatment, purification by mechanical surface stripping, purification by electrochemical etching, and a combination of these purification methods, wherein cleaning by electrochemical etching is most preferred. This preferred embodiment is a 41st embodiment of the method according to the invention, which is preferably dependent on an embodiment selected from the 1st to 40th embodiment.

|2b| contribution to achieving at least one of the objects mentioned in the beginning is also made by a 1st embodiment of a layered body 1 obtainable by, preferably obtained by, the method according to the invention for producing a layered body, preferably by the method according to an embodiment selected from the 1st to 41st embodiment.

|2| According to a preferred embodiment of the layered body 1, the latter has at least one of the following properties:
(α) a charge storage capacity determined according to the test method described herein, measured in cathodic phase, in a range of −10 to −100 mC/cm², preferably in a range of −8 to −80 mC/cm² and most preferably in a range of −4 to −60 mC/cm²;
(β) an impedance |Z| determined according to the test method described herein, at a frequency of 1 Hz, in a range of 0.3 to 20 kOhm, preferably in a range of 0.4 to 10 kOhm and most preferably in a range of 0.5 to 6 kOhm;
(γ) a thickness of the layer containing an electrically conductive polymer in a range of 100 to 3,000 nm, preferably in a range of 200 to 2,000 nm and most preferably in a range of 300 to 1,000 nm;
(δ) a homogeneity (dₘₐₓ-dₘᵢₙ)/dₘₑₐₙ of the thickness of the layer containing an electrically conductive polymer of less than 1.4, preferably of less than 0.8 and most preferably of less than 0.2, wherein dₘᵢₙ is the minimum thickness of the layer, dₘₐₓ is the maximum thickness of the layer and dₘₑₐₙ is the mean value of the thickness of the layer within the part of the electrically conductive substrate that is coated with the layer;
(ε) a surface roughness of the layer containing an electrically conductive polymer in a range of 10 to 1,000 nm, preferably in a range of 20 to 500 nm and most preferably in a range of 50 to 200 nm;
(ζ) a detachment, determined according to the test method described herein, of the layer containing an electrically conductive polymer of less than 50%, preferably less than 35%, and most preferably less than 20%, in each case based on the total weight of the layer in the layered body.

Layered bodies having the following properties or combinations of properties are particularly preferred according to the invention: (α), (β), (γ), (δ), (ε), (ζ), (α)(β), (α)(γ), (α)(γ), (α)(δ), (α)(ε), (α)(ζ), (β)(65 ), (β)(ε), (β)(ζ), (γ)(δ), (γ)ε), (γ)(ζ), (δ)(ε), (δ)(ζ), (ε)(ζ), (α)(β)(γ), (α)(β)(δ), (α)(β(249 ), (α)(β)(ζ) and (α)(β)(γ)(δ)(ε)(ζ), wherein (α)(β)(γ)(δ)(ε)(ζ) is most preferred. This preferred embodiment is a 2nd embodiment of the layered body 1 according to the invention, which is preferably dependent on the 1st embodiment.

|1c| A contribution to achieving at least one of the objects mentioned in the beginning is also made by a 1st embodiment of a layered body 2 comprising an electrically conductive substrate and a layer containing an electrically conductive polymer deposited on at least part of the surface of the electrically conductive substrate, wherein the layered body has at least one of the following properties:
(α) a charge storage capacity determined according to the test method described herein, measured in cathodic phase, in a range of −10 to −100 mC/cm², preferably in a range of −8 to −80 mC/cm² and most preferably in a range of −4 to 60 mC/cm²;
(β) an impedance |Z| determined according to the test method described herein, at a frequency of 1 Hz, in a range of 0.3 to 20 kOhm, preferably in a range of 0.4 to 10 kOhm and most preferably in a range of 0.5 to 6 kOhm;

(γ) a thickness of the layer containing an electrically conductive polymer in a range of 100 to 3,000 nm, preferably in a range of 200 to 2,000 nm and most preferably in a range of 300 to 1,000 nm;
(δ) a homogeneity (dₘₐₓ-dₘᵢₙ)/dₘₑₐₙ of the thickness of the layer containing an electrically conductive polymer of less than 1.4, preferably of less than 0.8 and most preferably of less than 0.2, wherein dₘᵢₙ is the minimum thickness of the layer, dₘₐₓ is the maximum thickness of the layer and dₘₑₐₙ is the mean value of the thickness of the layer within the part of the electrically conductive substrate that is coated with the layer;
(ε) a surface roughness of the layer containing an electrically conductive polymer in a range of 10 to 1,000 nm, preferably in a range of 20 to 500 nm and most preferably in a range of 50 to 200 nm;
(ζ) a detachment, determined according to the test method described herein, of the layer containing an electrically conductive polymer of less than 50%, preferably less than 35%, and most preferably less than 20%, in each case based on the total weight of the layer in the layered body.

Layered bodies having the following properties or combinations of properties are particularly preferred according to the invention: (α), (β), (γ), (δ), (ε), (ζ), (α)(β), (α)(γ), (α)(γ), (α)(δ), (α)(ε), (α)(ζ), (β)(γ), (β)(δ), (β)(ε), (β)(ζ), (γ)(δ), (γ)(ε), (γ)(ζ), (δ)(ε), (δ)(ζ) (ε)(ζ), (α)(β)(γ), (α)(β)(δ), (α)(β)(ε), (α)(β),(ζ), and (α)(β)(γ)(δ)(ε)(ζ), wherein (α)(β)(γ)(δ)(ε)(ζ) is most preferred.

|2c| According to a preferred embodiment of the layered body 2, the electrically conductive substrate is selected from the group consisting of gold, nickel, cobalt, iridium, titanium, tungsten, steel, platinum, silicon and alloys of at least two of these metals. This preferred embodiment is a 2nd embodiment of the layered body 2 according to the invention, which is preferably dependent on the 1st embodiment.

|3c| According to a further preferred embodiment of the layered body 2, the electrically conductive substrate is selected from the group consisting of gold and platinum. This preferred embodiment is a 3rd embodiment of the layered body 2 according to the invention, which is preferably dependent on the 2nd embodiment.

|4c| According to a further preferred embodiment of the layered body 2, the electrically conductive polymer is selected is from the group consisting of a polythiophene, a polypyrrole, a polyaniline or a combination of at least two thereof. This preferred embodiment is a 4th embodiment of the layered body 2 according to the invention, which is preferably dependent on an embodiment selected from the 1st to 3rd embodiment.

|5c| According to a further preferred embodiment of the layered body 2, the electrically conductive polymer is selected from the group consisting of poly(3,4-ethylenedioxythiophene) (PEDOT), poly(hydroxymethyl-EDOT), poly(EDOT-vinyl), poly(EDOT-allyl ether), poly(EDOT-COOH), poly(EDOT-MeOH), poly(EDOT-silane), poly(EDOT-acrylate), poly(EDOT-sulfonate), poly(EDOT-amine), poly(EDOT-amide), poly(sodium 4-[(2,3-dihydrothieno[3,4-b[]1,4]dioxin-2-yl)methoxy]butane-1-sulfonate) (PEDOT-S), poly(sodium 4-[(2,3-dihydrothieno[3,4-b][1,4]dioxin-2-yl)methoxy]butane-2-sulfonate) (PEDOT-S) and copolymers thereof. This preferred embodiment is a 5th embodiment of the layered body 2 according to the invention, which is preferably dependent on an embodiment selected from the 1st to 4th embodiment.

|6c| According to a further preferred embodiment of the layered body 2, the electrically conductive polymer is poly(3,4-ethylenedioxythiophene) (PEDOT). This preferred embodiment is a 6th embodiment of the layered body 2 according to the invention, which is preferably dependent on the 5th embodiment.

|7c| According to a further preferred embodiment of the layered body 2, the layered body is an electrode or part of an electrode for a medical device, particularly preferably an electrode or part of an electrode for detecting electrophysiological signals in a tissue or in a part of a tissue or for electrically stimulating a tissue or a part of a tissue, even more preferably an implantable electrode or part of an implantable electrode. In this context, it is also preferred that the coating with the electrically conductive polymer is arranged in the layered body 2 according to the invention such that, when used during a use of this layered body as an electrode or as part of an electrode for detecting electrophysiological signals in a tissue or in a part of a tissue or for electrically stimulating a tissue or a part of a tissue, the coating comes into contact, preferably into direct contact, with the tissue or the part of the tissue. This preferred embodiment is a 7th embodiment of the layered body 2 according to the invention, which is preferably dependent on an embodiment selected from the 1st to 6th embodiment.

|1d| A contribution to achieving at least one of the above-mentioned objects is also made by a medical device containing a layered body 1 according to the invention, preferably according to its 1st or 2nd embodiment, or a layered body 2 according to the invention, preferably according to an embodiment selected from the 1st to 7th embodiment, as an electrode or part of an electrode, particularly preferably as an electrode or part of an electrode for detecting electrophysiological signals in a tissue or in a part of a tissue or for electrically stimulating a tissue or a part of a tissue, even more preferably as an implantable electrode or part of an implantable electrode.

|1e| A contribution to achieving at least one of the objects mentioned in the beginning is also made by the use of a layered body 1 according to the invention, preferably according to its 1st or 2nd embodiment, or of a layered body 2 according to the invention, preferably according to an embodiment selected from the 1st to 7th embodiment, as an electrode or part of an electrode in a medical device. Particularly preferably, the layered body 1 or the layered body 2 is used as an electrode or part of an electrode for detecting electrophysiological signals in a tissue or in a part of a tissue or for electrically stimulating a tissue or a part of a tissue, even more preferably as an implantable electrode or part thereof, for example for determining a physiological state in a living organism, preferably in the human body.

### Method Step a)

In method step a) of the method according to the invention, an electrically conductive substrate is provided. Suitable electrically conductive substrates are metals, ceramics, polymers, composite materials and the like. The substrate surface can be a carbon nitride, a carbon fabric, a carbon paper, an electrode printed with carbon screen printing, a carbon black, a carbon powder, a carbon fiber, a carbon nanotube, a diamond-coated conductor, a glass-like carbon, a mesoporous carbon, a graphite or a combination of at least two of these materials. The substrate surface may contain a non-metallic inorganic material, such as a metal oxide, a metal nitride, a ceramic, a metalloid or a combination of at least two thereof. The non-metallic inorganic material may comprise, for example, a metalloid selected from the group consisting of silicon, carbon and a combination thereof. The substrate surface can contain a metal oxide, for example an oxide of aluminum, titanium, zirconium, hafnium, tantalum, molybdenum, chromium, nickel, tungsten, rhenium, iridium or a combination of at least two thereof. The substrate surface may consist of a ceramic, such as a silicon nitride, a titanium nitride, a silicon carbide, a silicon oxide, a calcium phosphate, an indium tin oxide, or a combination thereof. The substrate surface may contain a metal selected from the group consisting of a noble metal, a transition metal, or a combination thereof. The metal may be selected, for example, from the group consisting of gold, platinum, palladium, iridium, osmium, rhodium, titanium, niobium, tantalum, tungsten, ruthenium, magnesium, iron, and a combination thereof. The substrate surface can also contain a non-noble metal which is selected from the group consisting of titanium, tantalum, and a combination thereof. The substrate surface can contain a metal alloy which contains, for example, at least one noble metal and at least one transition metal. The metal alloy can contain, for example,
nickel and titanium;
nickel and cobalt;
cobalt and chromium;
niobium and titanium,
or a combination of at least two of these alloys.

The metal alloy may also be a stainless steel alloy, preferably selected from the group consisting of stainless steel 304L, stainless steel 316L, stainless steel 316LVM, and a combination of at least two thereof. The metal alloy may also be a cobalt-nickel-chromium alloy, preferably selected from the group consisting of MP35N, 35NLT, and a combination of at least two thereof. The metal alloy can also contain the titanium alloy Ti-6A1-4V. The metal alloy can also contain nitinol, e.g., Ni55 Ti45. The metal alloy can also contain niobium-titanium, e.g., Nb56 Ti44.

Furthermore, the electrically conductive substrate provided in method step a) of the

method according to the invention can have any shape, including, but not limited to, the shape of a cuboid, a cylinder, a sphere, a pyramid, a tube, a disk, a mesh, a wire, or a combination of these shapes. Furthermore, the electrically conductive substrate can be an electrode or part of an electrode, preferably an implantable electrode, for example for determining a physiological state in a living organism, preferably in the human body.

The electrically conductive substrate provided in method step a) can be purified and/or subjected to further surface modifications, such as a roughening, prior to method steps b) and c), i.e., prior to electrodeposition of the electrically conductive polymer. In this context, reference is made to the statements in column 11, lines 21-65 of U.S. Pat. 10,800,931 B2.

### Method Step b)

In method step b) of the method according to the invention, at least part of the surface of the electrically conductive substrate is contacted with a composition containing i) at least one monomer suitable for producing an electrically conductive polymer, ii) at least one solvent and optionally iii) at least one crosslinker, preferably a photoreactive crosslinker.

The crosslinker iii) may contain an anionic photoreactive hydrophilic polymer, an anionic photoreactive crosslinking agent, a photoreactive and uncharged hydrophilic polymer, or a combination of at least two of these components. These photoreactive compounds are preferably characterized in that they have a latent photoreactive group or a photoreactive group.

The terms “latent photoreactive group” and “photoreactive group” are used interchangeably herein and each refer to a chemical entity that is sufficiently stable to remain in an inactive state (i.e., the ground state) under normal storage conditions, but which can transition to an active state when exposed to a suitable energy source. Unless otherwise specified, references to photoreactive groups preferably also apply to the reaction products of the photoreactive groups.

Photoreactive groups react to specific external stimuli by forming active groups that form covalent bonds with adjacent chemical structures. For example, a photoreactive group can be activated and a hydrogen atom can be cleaved from an alkyl group. A covalent bond can be formed between the compound with the photoreactive group and the compound with a C—H bond. Suitable photoreactive groups are described, for example, in U.S. Pat. No. 5,002,582. The photoreactive groups can be selected to react to different parts of the actinic radiation. For example, groups can be selected to be activated either by ultraviolet (UV) or visible (VIS) radiation.

Suitable photoreactive groups are, for example, azides, diazos, diazidines, ketones and quinones. When electromagnetic energy is absorbed, the photoreactive groups generate active species such as free radicals, including, e. g., nitrenes, carbenes and excited states of ketones. The photoreactive group can consist of an aryl ketone, such as acetophenone, benzophenone, anthrone and anthrone-like heterocycles (i.e., heterocyclic analogues of anthrone, such as those with N, O or S in the 10 position), or their substituted (e. g., ring-substituted) derivatives. Examples of aryl ketones are heterocyclic derivatives of anthrone, including acridone, xanthone, and thioxanthone, and their ring-substituted derivatives. Other suitable photoreactive groups are quinones, such as anthraquinone. The functional groups of such aryl ketones can undergo several activation/inactivation/reactivation cycles. Benzophenone, for example, can be photochemically excited with the initial formation of an excited singlet state which transitions to the triplet state. The excited triplet state can be inserted into carbon-hydrogen bonds by abstraction of a hydrogen atom (e.g., from a polymeric coating) and can thus form a radical pair. The subsequent collapse of the radical pair results in the formation of a new carbon-carbon bond. If no reactive bond (e. g., carbon/hydrogen) is available, the excitation of the benzophenone group induced by ultraviolet light is reversible and the molecule returns to the ground state once the energy source has been removed. Photoreactive aryl ketones such as benzophenone and acetophenone can undergo several reactivations in water and therefore provide a higher coating efficiency.

The azides form a further class of photoreactive groups and comprise aryl azides (C₆R₅N₃), such as phenyl azide and 4-fluoro-3-nitrophenyl azide; acyl azides (—CO—N₃), such as benzoyl azide and p-methylbenzoyl azide; azidoformates (—O—CO—N₃), such as ethyl azidoformate and phenyl azidoformate; sulfonyl azides (—SO₂—N₃), such as benzenesulfonyl azide; and phosphoryl azides (RO)₂PON₃, such as diphenylphosphoryl azide and diethylphosphoryl azide. Diazo compounds form a further class of photoreactive groups and comprise diazoalkanes (−CHN₂), such as diazomethane and diphenyldiazomethane; diazoketones (—CO—CHN₂), such as diazoacetophenone and 1-trifluoromethyl-l-diazo-2-pentanone; diazoacetates (—O—CO—CHN₂), such as t-butyldiazoacetate and phenyldiazoacetate; and beta-keto-alpha-diazoacetates (—CO—CN₂—CO—O—), such as, e.g., t-butyl-alpha-diazoacetoacetate. Other photoreactive groups are the diazirines (—CHN₂), such as, e. g., 3-trifluoromethyl-3-phenyldiazirine; and ketenes (—CH═C═O), such as, e.g., ketene and diphenylketene.

It is particularly preferred to use the composition “Amplicoat®” available from Heraeus Medical Components, Germany, in method step b).
Method Step c)

In method step c) of the method according to the invention, an electrical potential is applied between the electrically conductive substrate and the composition. This electrochemical polymerization step is generally carried out in an electrodeposition device comprising a container into which at least two electrodes are introduced. The electrodeposition device preferably comprises the electrically conductive substrate as a working electrode, a counter electrode with approximately 10 times the surface of the working electrode, which can consist of platinum, platinized titanium or platinized niobium, and optionally a reference electrode which can be, for example, an Ag/AgCl or calomel reference electrode saturated with KCl. The container of such an electrodeposition device is filled with a composition containing i) the at least one monomer suitable for producing an electrically conductive polymer, ii) the at least one solvent and optionally iii) the at least one crosslinker, particularly preferably with an “Amplicoat®” composition, and the electrically conductive substrate as the working electrode is immersed at least partially into this composition. Polymerization of the monomer i) is then initiated by applying an electrical potential between the working electrode and the composition.
Method Step d)

In method step d) of the method according to the invention, the layer obtained in method step c) and containing an electrically conductive polymer is optionally contacted with at least one crosslinker, preferably a photoreactive crosslinker. This method step d) preferably takes place only if the composition used in method step b) did not contain any crosslinker or photoreactive crosslinker.

Contacting can be done by immersing the coated electrically conductive substrate obtained in method step c) into a composition containing a crosslinker, or by applying a composition containing a crosslinker to the coated electrically conductive substrate obtained in method step c) by spraying, by printing, by applying by means of a paintbrush, applying by means of a brush, applying by means of a felt or applying by means of a cloth, by vapor deposition or by a combination of at least two of these methods. Optionally, solvents present in the composition can be removed preferably by evaporation after the contacting and before carrying out the photoreaction in method step e).

Method Step e)

In method step e) of the method according to the invention, electromagnetic radiation is applied to the layer containing the electrically conductive polymer in order to achieve a photoreaction of the at least one photoreactive crosslinker iii) (if and insofar as the latter was a constituent of the composition used in method step b)). Preferably, the electromagnetic radiation applied is UV light in a wavelength range of 260 to 400 nm. The duration of irradiation with UV light is preferably in a range of 1 sec to 15 min, preferably in a range of 2 sec to 10 min, and particularly preferably in a range of 3 sec to 1 min (per coated area).

The invention will now be explained in more detail with reference to non-limiting figures and examples.

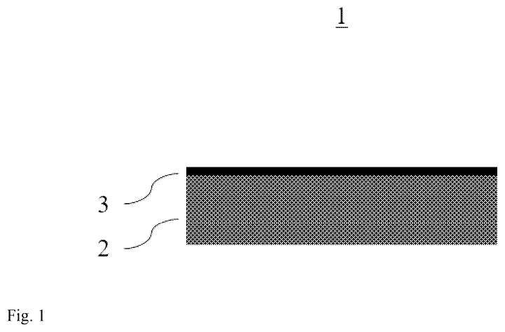

FIG. 1 shows a layered body 1 according to the invention, comprising an electrically conductive substrate 2. At least part of the surface of the electrically conductive substrate 2 is coated with a layer 3 containing an electrically conductive polymer. Preferably, the layered body is an implantable electrode or an implantable sensor or at least part of an implantable electrode or of an implantable sensor.

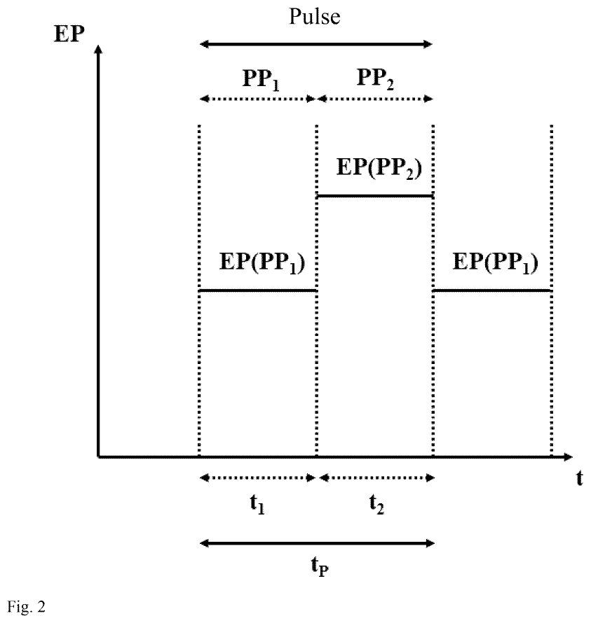

FIG. 2 shows the profile of the electrical potential EP as a function of time t in the case of a pulse potentiostatic polymerization process. In this process, polymerization of the monomer i) suitable for producing an electrically conductive polymer takes place in method step c) during a pulse phase PP having successive pulses. Each pulse comprises a partial pulse phase PP₁ with a phase duration t₁ and a partial pulse phase PP₂ with a phase duration t₂, wherein partial pulse phase PP₁ is characterized by an electrical potential EP(PP₁) and by a current density SD(PP₁) guided through the electrically conductive substrate 2, and partial pulse phase PP₂ is characterized by an electrical potential EP(PP₂) and by a current density SD(PP₂) guided through the electrically conductive substrate 2. In the pulse potentiostatic polymerization process shown in FIG. 2, EP(PP₁)<EP(PP₂). This method is further characterized in that, during partial pulse phase PP₁ and partial pulse phase PP₂, the electrical potential is constant within the relevant partial pulse phase. Although each pulse in FIG. 2 starts with a partial pulse phase PP₁, it is also possible to have configurations of the method in which each pulse starts with a partial pulse phase PP₂. With the pulse potentiostatic polymerization process shown in FIG. 2, each pulse consists of partial pulse phase PP₁ and partial pulse phase PP₂, so that the total duration tₚ of a pulse equals the sum of t₁ and t₂. However, pulse potentiostatic polymerization processes in which each pulse consists of more than two partial pulse phases that differ in terms of the constantly applied electrical potential are also conceivable. The duration of a relevant partial pulse phase PP₁ or PP₂ and the number of pulses in pulse phase PP depend in particular on the thickness to be achieved for layer 3 containing the electrically conductive polymer, which layer is to be deposited on the electrically conductive substrate 2.

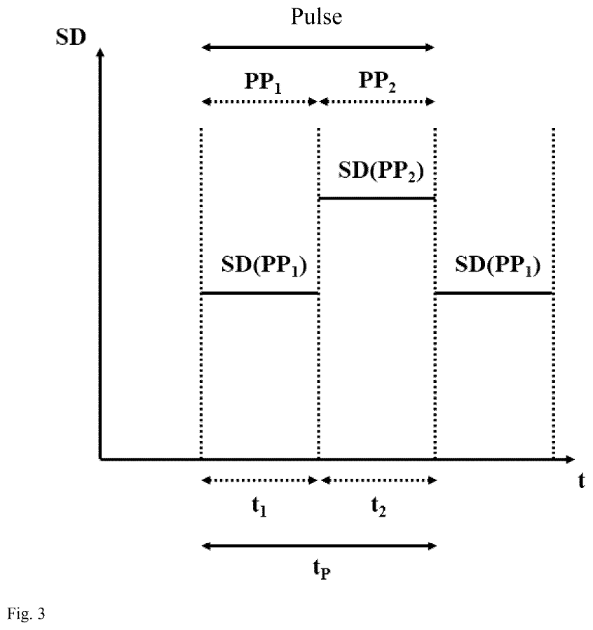

FIG. 3 shows the profile of the current density SD guided through the electrically conductive substrate 2 as a function of time t in the case of a pulse galvanostatic polymerization process. In contrast to the pulse potentiostatic polymerization process shown in FIG. 2, the pulse galvanostatic polymerization process is characterized in that, during partial pulse phase PP₁ and partial pulse phase PP₂, the current density guided through the substrate is constant within the relevant pulse phase in each case.

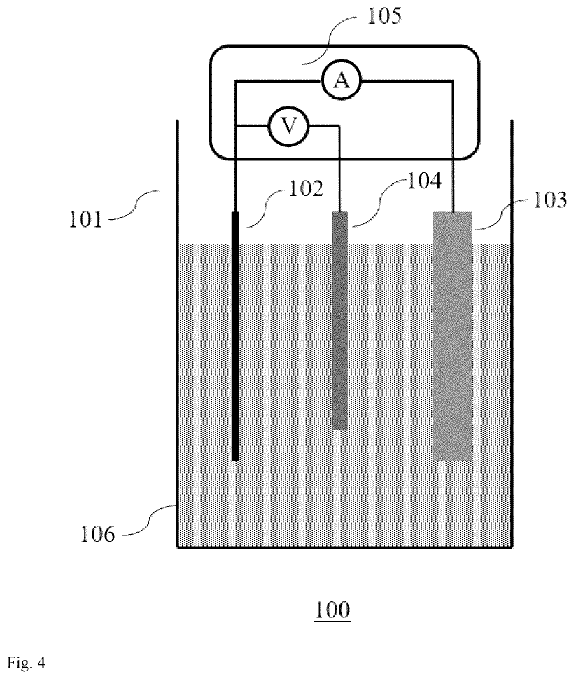

FIG. 4 shows an electrodeposition device 100 comprising a container 101 in which the method according to the invention can be carried out. In addition to the container 101, this device comprises at least two electrodes 102 and 103 which are introduced into the container 101. The electrodeposition device 100 preferably comprises the electrically conductive substrate as a working electrode 102, a counter electrode 103 with approximately 10 times the surface of the working electrode, which can consist of platinum, platinized titanium or platinized niobium, and optionally a reference electrode 104 which can be, for example, an Ag/AgCl or calomel reference electrode. The container 101 of such an electrodeposition device 100 is filled with a composition containing i) the at least one monomer suitable for producing an electrically conductive polymer, ii) the at least one solvent and optionally iii) the at least one crosslinker, particularly preferably with an “Amplicoat®” composition, and the electrically conductive substrate as the working electrode 102 is immersed at least partially into this composition. Polymerization of the monomer i) is then initiated by applying an electrical potential between the working electrode 102 and the composition.

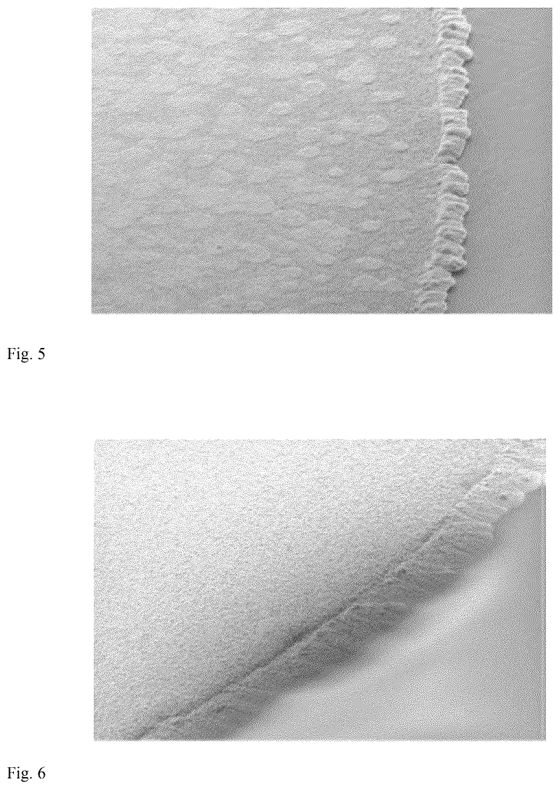

FIG. 5 shows an electron photomicrograph of the PEDOT-coated gold disk obtained in Example 1 at an image scale of 500:1.

FIG. 6 shows an electron photomicrograph of the PEDOT-coated gold disk obtained in Example 2 at an image scale of 500:1.

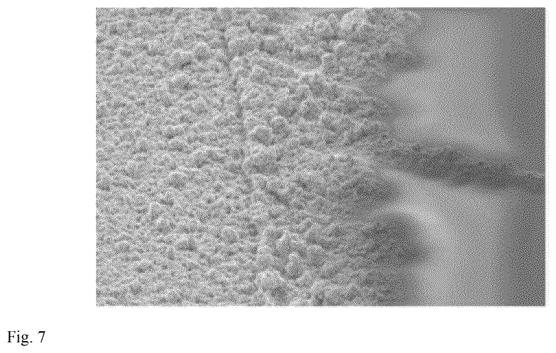

FIG. 7 shows an electron photomicrograph of the PEDOT-coated gold disk obtained in the comparative example at an image scale of 1000:1.

### TEST METHODS

### Determination of Charge Storage Capacity

The charge storage capacity of the layered body was determined by means of cyclic voltammetry using a BIO-LOGIC VMP3 potentiostat/galvanostat, as described in U.S. Pat. No. 10,800,931 B2.

### Determination of Impedance |Z|

The impedance |Z| of the layered body was determined by means of impedance spectrometry using a BIO-LOGIC VMP3 potentiostat/galvanostat, as described in U.S. Pat. No. 10,800,931 B2.

### Determination of the Thickness of Layer 3

The thickness of layer 3 was determined by means of scanning electron microscopy.

### Determination of the Homogeneity of the Thickness of Layer 3

The homogeneity of the thickness of layer 3 was determined by means of scanning electron microscopy.

### Determination of the Roughness of Layer 3

The surface roughness of layer 3 was determined by means of scanning electron microscopy.

Determination of the Detachment of Layer 3

All coated electrodes were tested for the resulting mechanical stability of the film for the respective electrode substrates. For this purpose, the wiping test method was used in which a swab with a foam tip (Texwipe TX751B) is used as a contact probe. The contact pressure was set by loading the fixation stick of the swab with weights. The weight load by the swab was 100 g for a contact surface of 1 mm². The surface relates to the contact surface of the swab at the lowest possible contact pressure. The same operator performed ten forward and backward movements at constant speed with the swab. After weight loading, the resulting integrity of the electrode surface was visually analyzed and quantified as the percentage loss of the Amplicoat® film compared to the initial coating (100%).

### EXAMPLES

All electrodes were cleaned in a similar manner prior to electrodeposition, wherein the following order was complied with:
a) Polishing with aluminum oxide paste (10 μm and 1 μm particle size) and diamond paste (0.1 μparticles) on fabric pads.
b) Removal of the remaining particles by ultrasonic cleaning in a 2% Alconox solution at 40° C.;
c) Repeated washing with water and isopropanol.

The composition “Amplicoat®” available from Heraeus Medical Components, Germany, was used as the composition containing i) at least one monomer suitable for producing an electrically conductive polymer, ii) at least one solvent, and iii) at least one photoreactive crosslinker.

### Example 1 (Pulse Potentiostatic Method)

In a device as shown in FIG. 4, the following parameters were used to deposit PEDOT from an “Amplicoat®” composition onto a disk-shaped gold electrode:

| Number of pulses | 24 | |
|---|---|---|
| Duration t₁ of partial pulse phase PP₁ | 10 | sec |
| electrical potential EP(PP₁) | 0.5 | V |
| Duration t₂ of partial pulse phase PP₂ | 5 | sec |
| electrical potential EP(PP₂) | 1.1 | V |
| Duration tₒ of the initiation phase | 10 | sec |
| electrical potential EP₀ in the equilibration phase | 0.5 | v |

FIG. 5 shows an electron photomicrograph of the PEDOT-coating obtained in Example 1 at a scale of 500:1.

### Example 2 (Pulse Galvanostatic Method)

In a device as shown in FIG. 4, the following parameters were used to deposit PEDOT from an “Amplicoat®” composition onto a disk-shaped gold electrode:

| Number of pulses | 24 | |
|---|---|---|
| Duration t₁ of partial pulse phase PP₁ | 10 | sec |
| Current density SD(PP₁) | 0 | mA/cm² |
| Duration t₂ of partial pulse phase PP₂ | 5 | sec |
| Current density SD(PP₂) | 1.3 | mA/cm² |
| Duration tₒ of the initiation phase | 10 | sec |
| Current density SD₀ in the equilibration phase | 0 | mA/cm² |

FIG. 6 shows an electron photomicrograph of the PEDOT-coating obtained in Example 2 ata scale of 500:1.

### Comparative Example (Continuous Galvanostatic Method)

In a device as shown in FIG. 4, PEDOT from an “Amplicoat®” composition was deposited onto a disk-shaped gold electrode at a constant current density of 1.3 mA/cm² over a period of 2 minutes. FIG. 7 shows an electron photomicrograph of the PEDOT-coating obtained in this comparative example at a scale of 1000:1.

The following was determined in respect of the layered body obtained in Examples 1 and 2 and in the comparative example: the layer thickness of the PEDOT layer, its homogeneity, the film morphology (via the electron photomicrographs shown in FIGS. 5, 6 and 7) and adhesion of the PEDOT layers to the substrate.

| | | | Comparative |
|---|---|---|---|
| | Example 1 | Example 2 | example |
| Layer thickness | 1.3 μm | 600 nm | 400-900 nm |
| Homogeneity of the layer | + | + | − |
| thickness | | | |
| Film morphology | + | ++ | − |
| Adhesion | + | ++ | − |

### LIST OF REFERENCE SIGNS

1 Layered body
2 Electrically conductive substrate
3 Layer containing an electrically conductive polymer
100 Electrodeposition device
101 Container
102 Working electrode
103 Counter electrode
104 Reference electrode

1. A method for producing a layered body, wherein the layered body comprises an electrically conductive substrate, the surface of which is at least partially coated with a layer containing an electrically conductive polymer, the method comprising the following method steps:
a) providing the electrically conductive substrate;
b) contacting at least a part of the surface of the electrically conductive substrate with a composition containing
i) at least one monomer suitable for producing an electrically conductive polymer,
ii) at least one solvent,
iii) optionally at least one crosslinker, preferably a photoreactive crosslinker;
c) applying an electrical potential between the electrically conductive substrate and the composition such that the at least one monomer i) polymerizes to form an electrically conductive polymer, and the electrically conductive polymer formed is deposited in the form of a layer containing this polymer on at least a part of the surface of the electrically conductive substrate;
wherein applying the electrical potential comprises a pulse phase PP having successive pulses, wherein each pulse of the pulse phase PP comprises a partial pulse phase PP₁ having a phase duration t₁ and a partial pulse phase PP₂ having a phase duration t₂,
wherein the partial pulse phase PP₁ is characterized by an electrical potential EP(PP₁) and by a current density SD(PP₁) guided through the electrically conductive substrate;
wherein the partial pulse phase PP₂ is characterized by an electrical potential EP(PP₂) and by a current density SD(PP₂) guided through the electrically conductive substrate;
wherein EP(PP₁)<EP(PP₂) and/or SD(PP₁)<SD(PP₂);
d) optionally contacting the layer containing an electrically conductive polymer, which layer was obtained in method step c), with at least one crosslinker, preferably a photoreactive crosslinker;
e) optionally applying electromagnetic radiation to the layer containing the electrically conductive polymer in order to cause a photoreaction of the at least one photoreactive crosslinker iii).

2. The method according to claim 1, wherein EP(PP₁)<EP(PP₂) and wherein, during partial pulse phase PP₁ and partial pulse phase PP₂, the electrical potential is constant within the relevant partial pulse phase.

3. The method according to claim 1, wherein the electrical potential EP(PP₁) during partial pulse phase PP₁ is in a range of 0.2 to 0.8 V, and the electrical potential EP(PP₂) during partial pulse phase PP₂ is in a range of 0.8 to 1.4 V.

4. The method according to claim 2, wherein t₁ is in a range of 1 to 15 sec, and t₂ is in a range of 1 to 10 sec.

5. The method according to claim 1, wherein SD(PP₁)<SD(PP₂) and wherein, during partial pulse phase PP₁ and partial pulse phase PP₂, the current density guided through the electrically conductive substrate is constant within the relevant partial pulse phase.

6. The method according to claim 1, wherein the amount of the current density |SD(PP₁)| guided through the electrically conductive substrate during partial pulse phase PP₁ is less than 0.4 mA/cm², and the amount of the current density |SD(PP₂)| guided through the electrically conductive substrate during partial pulse phase PP₂ is in a range of 0.9 to 1.7 mA/cm².

7. The method according to claim 5, wherein t₁ is in a range of 1 to 15 sec, and t₂ is in a range of 1 to 10 sec.

8. The method according to claim 1, wherein pulse phase PP comprises at least 2 pulses.

9. The method according to claim 1, wherein the individual pulses of pulse phase PP follow one another such that partial pulse phase PP₁ is followed by partial pulse phase PP₂ and partial pulse phase PP₂ is followed by partial pulse phase PP₁.

10. The method according to claim 1, wherein the pulse phase PP has a total duration of 30 sec to 20 min.

11. The method according to claim 1, wherein the electrically conductive substrate is selected from the group consisting of gold, nickel, cobalt, iridium, titanium, tungsten, steel, platinum, silicon and alloys of at least two of these metals.

12. The method according to claim 1, wherein the monomer i) suitable for producing an electrically conductive polymer is selected from the group consisting of 3,4-ethylenedioxythiophene or a derivative thereof, pyrrole or a derivative thereof, aniline or a derivative thereof, or a combination of at least two of these monomers.

13. The method according to claim 12, wherein the monomer i) suitable for producing an electrically conductive polymer is 3,4-ethylenedioxythiophene or a derivative thereof.

14. The method according to claim 1, wherein the crosslinker iii) comprises at least one anionic photoreactive hydrophilic polymer containing polyacrylamide and photoreactive groups.

15. The method according to claim 14, wherein the at least one anionic photoreactive hydrophilic polymer has sulfonate groups.

16. The method according to claim 1, wherein the crosslinker iii) contains at least one anionic photoreactive crosslinking agent comprising sulfonate groups, carboxylate groups, phosphonate groups, phosphate groups or combinations of these groups.

17. The method according to claim 1, wherein the crosslinker iii) contains at least one photoreactive and uncharged hydrophilic polymer.

18. The method according to claim 17, wherein the at least one photoreactive and uncharged hydrophilic polymer is a photoreactive 1-vinyl-2-pyrrolidone derivative.

19. The method according to claim 1, wherein the solvent ii) is an aprotic organic solvent, a polar organic solvent or a mixture thereof.

20. The method according to claim 19, wherein the solvent ii) is selected from the group consisting of acetonitrile, dichloromethane, dimethyl sulfoxide, acetone, dimethylformamide, isopropanol, methanol, ethanol, water and mixtures of at least two of these solvents.

21. The method according to claim 1, wherein the composition used in method step b) contains a surface-active compound iv).

21. he method according to claim 21, wherein the surface-active compound iv) is selected from the group consisting of poloxamers, polyoxyethylene alkyl ethers, polysorbitan, polyoxyethylene derivatives of sorbitan monolaurate and mixtures of at least two of these compounds.

23. The method according to claim 1, wherein the electromagnetic radiation applied in method step e) is UV light in a wavelength range of 260 to 400 nm.

24. A layered body obtainable by the method according to claim 1.

25. A layered body comprising an electrically conductive substrate and a layer containing an electrically conductive polymer deposited on at least part of the surface of the electrically conductive substrate, wherein the layered body has at least one of the following properties:
(α) a charge storage capacity determined according to the test method described herein, measured in cathodic phase, in a range of −10 to −100 mC/cm²;
(β) an impedance |Z| determined according to the test method described herein, at a frequency of 1 Hz, in a range of 0.3 to 20 kOhm;
(γ) a thickness of the layer containing an electrically conductive polymer in a range of 100 to 3,000 nm;
(δ) a homogeneity (dₘₐₓ-dₘᵢₙ)/dₘₑₐₙ of the thickness of the layer containing an electrically conductive polymer of less than 1.4, wherein dₘᵢₙ is the minimum thickness of the layer, dₘₐₓ is the maximum thickness of the layer, and dₘₑₐₙ is the mean value of the thickness of the layer within the part of the electrically conductive substrate that is coated with the layer;
(ε) a surface roughness of the layer containing an electrically conductive polymer in a range of 10 to 1,000 nm;
(ζ) a detachment determined according to the test method described herein of the layer containing an electrically conductive polymer of less than 50% based on the total weight of the layer in the layered body.

26. The layered body according to claim 24, wherein the layered body is an electrode or part of an electrode for a medical device.

27. A medical device containing a layered body according to claim 24 as an electrode or part of an electrode.

28. Use of a layered body according to claim 24 as an electrode or part of an electrode in a medical device.
